(19) United States
(12) Patent Application Publication
(10) Pub. No.: US 2011/0053263 A1
(43) Pub. Date: Mar. 3, 2011

(54) Multipotent Cancer Stem Cell Lines and Method for Producing the Same

(75) Inventors: Dong-Young Noh, Seoul (KR); Won-shik Han, Seoul (KR); Eunyoung Ko, Seoul (KR); Jong Bin Kim, Gyeonggi-do (KR); Kyung-Min Lee, Seoul (KR)

(73) Assignee: SNU INDUSTRY FOUNDATION, Seoul, KR (KR)

(21) Appl. No.: 12/918,141

(22) PCT Filed: Feb. 18, 2009
(86) PCT No.: PCT/KR09/00783

§ 371 Date: Nov. 18, 2010

(30) Foreign Application Priority Data
Feb. 18, 2008 (KR) 10-2008-0014498

(51) Int. Cl. C12N 5/095

(52) U.S. Cl. 435/325

(57) ABSTRACT

Provided is a multipotent cancer stem cell line derived from breast-cancer tissue and expressing a breast cancer stem cell marker. Also provided is a method for producing a multipotent cancer stem cell line, including (1) isolation of breast-cancer cells from previously extracted breast cancer tissue, (2) primary culture of the isolated breast cancer cells in a suspended state in a medium for suspension culture, (3) recovery of the cells in the suspended state from the primary culture, and (4) production of a multipotent cancer stem cell line by subculturing the recovered cells a predetermined number of times or more in a suspended state in the medium for suspension culture.

### TECHNICAL FIELD

The present invention relates to a multipotent cancer stem cell line and a method for producing the same, and more particularly, a multipotent stem cell line derived from breast cancer tissue and expressing a breast cancer stem cell marker and a method for producing the same, including: (1) isolation of breast cancer cells from previously extracted breast cancer tissue; (2) primary culture of the isolated breast cancer cells in a suspended state in a medium for suspension culture; (3) recovery of the cells in the suspended state from the primary culture; and (4) production of a multipotent cancer stem cell line by subculturing the recovered cells a predetermined number of times or more in a suspended state in the medium for suspension culture.

### BACKGROUND ART

Despite continuous attempts, various cancer therapies including surgical therapy, radiotherapy, immunotherapy and gene therapy have not improved therapeutic efficiency for patients with tumors. It is assumed that tumors are caused by transformation of normal stem cells in the bone marrow, but it has been shown that the presence of adult stem cells in some other organs including the epidermal breasts and brain can initiate tumor stem cells capable of forming solid tumors. The fact that a minority of cells stimulating non-uniform tumor formation are present in breast cancer and brain tumors indicates that tumor stem cells are generated from breast cells or neural stem cells.

Cancer is understood as being a state of dedifferentiation, since it shows less differentiated cell structures similar to fetus tissue. Today, pathologists still mention a “degree of dedifferentiation” in terms of tumor grading. Insufficient differentiation leads to an unfavorable diagnosis when compared to sufficient differentiation. Extrinsic factors such as chemicals or viruses induce dedifferentiation of mature adult cells, which are then differentiated into the shape of a stem cell and cause cancer.

It is widely known that changes in growth-control function by genetic transformation lead to abnormal growth as in cancer. In terms of self-renewal, genetics limits proliferation of stem cells in normal tissue. Altering the control of self-renewal is considered as a key factor in the development of cancer based on the fact that some paths involved in carcinogenesis play important roles for determining the self-renewal of normal stem cells.

There is a growing body of evidence showing that cancer tissue can contain their own stem cells. Many cancers, similar to normal tissue, are maintained by systemized cell populations consisting of a hierarchy including slowly-dividing stem cells, rapidly-dividing temporary proliferating cells and differentiated cells.

It is unclear whether or not a cell that originates cancer stem cells is derived from normal stem cells. Malignant gliomas sometimes include both non-differentiated and differentiated stem cell populations, or include cells expressing a glial mark and nestin. This means that the malignant gliomata may include neural progenitors having various potentials. The presence of a subpopulation, a small particular biological group, of slowly differentiated cancer stem cells may be an important factor in the recurrence of cancer, since the cells survive after radiation or treatment with cytotoxic drug cancer cells while most cancer cells are killed. Of the cancer cells that survive after treatment, the living cells are cancer stem cells. Such tumor stem cells have resistance to treatment and are essential in the malignance of cancer. Some rare kinds of tumor stem cells cause malignance in cancer tissue, and thus the purpose of cancer treatment is to identify such tumor stem cells and develop a treatment targeting the same.

### DISCLOSURE

### Technical Problem

The present inventors identified multipotent cancer stem cells from breast cancer tissue and expressing breast cancer stem cell markers while searching for cancer stem cells in order to provide a cornerstone for novel cancer treatment. The present inventors also found that multipotent cancer stem cells have multipotency in that they express an epithelial cell marker, a neural cell marker and a mesenchymal cell marker, have resistance to antibiotics, and cause a tumor when transplanted into an individual. Thus, the present invention was completed.

The present invention is directed to a multipotent cancer stem cell line derived from breast cancer tissue expressing a breast cancer stem cell marker and capable of being cultured in a suspended state.

The present invention is also directed to a method for producing a multipotent cancer stem cell line, including: (1) isolation of breast cancer cells from previously extracted breast cancer tissue; (2) primary culture of the isolated breast cancer cells in a suspended state in a medium for suspension culture; (3) recovery of the cells in the suspended state from the primary culture; and (4) production of a multipotent cancer stem cell line by subculturing the recovered cells a predetermined number of times or more in a suspended state in the medium for suspension culture.

### Technical Solution

The present invention relates to a multipotent cancer stem cell line derived from breast cancer tissue and expressing a breast cancer stem cell marker.

The multipotent cancer stem cell line according to the present invention may be derived from breast cancer tissue, though preferably a sarcoma. A sarcoma refers to a malignant tumor derived from mesenchymal tissue generated in connective tissue, bones or muscles. Generally, a sarcoma is a malignant tumor and difficult to treat compared to a carcinoma originating from an epithelial cell. Thus, the multipotent cancer stem cell line of the present invention derived from the sarcoma may be used to develop a treatment for cancer, which may be derived from the sarcoma but is difficult to treat by conventional methods.

The multipotent cancer stem cell line according to the present invention may express a breast cancer stem cell marker. Particularly, examples of breast cancer stem cell markers may include CD24 and CD44, which are tumor stem cell markers. These cell markers exhibit the CD24^{low/-}CD^{44high} pattern.

The multipotent cancer stem cell line according to the present invention may have multipotency. The multipotent cancer stem cell line according to the present invention may be derived from breast cancer tissue but may cause other types of cancer other than breast cancer. Thus, the multipotent cancer stem cell line according to the present invention may be used to provide therapies for various cancers including breast cancer.

The multipotent cancer stem cell line according to the present invention may express at least one selected from the group consisting of markers of an epithelial cell, a neural cell and a mesenchymal cell. The multipotent cancer stem cell line may express Vimentin and Muc1 as the epithelial cell markers, nestin and Tuj-1 as the neural cell markers and fibronectin as the mesenchymal cell marker.

In addition, the multipotent cancer stem cell line according to the present invention may have resistance to an anticancer drug, and particularly express ABCG2 as an anticancer drug-resistant protein.

The multipotent cancer stem cell line according to the present invention may be produced by a method, including: (1) isolation of breast cancer cells from previously extracted breast cancer tissue; (2) primary culture of the isolated breast cancer cells in a suspended state in a medium for suspension culture; (3) recovery of the cells in the suspended state from the primary culture; and (4) production of a multipotent cancer stem cell line by subculturing the recovered cells a predetermined number of times or more in a suspended state in the medium for suspension culture.

In step (1), an isolated, preferably single cancer cell, may be obtained by incising previously extracted breast cancer tissue. In one aspect, the cancer tissue may be finely cut by physical means such as a homogenizer, a mortar, a blender, a surgical mass, a syringe, forceps or an ultrasonic apparatus. In another aspect, the cancer tissue may be finely cut by treating the cancer tissue with an enzyme. Here, examples of the enzymes used herein may include, but are not limited to, serine proteases including neutral protease, trypsin, chimotrypsin, thermolysin, elastases and collagenases. In still another aspect, the fine-cutting may be carried out using both the physical means and the enzyme treatment described above.

In step (2), the previously isolated cancer cells may be primarily cultured in a medium for suspension culture. A medium composed of DMEM, F12, B27 supplement and growth factors (e.g., EGF, PDGF, VEGF, FGF, IGF, LIF, etc.) may be suitable for suspension culture. If necessary, the medium may contain components such as cytokines (e.g., insulin, estradiol, interleukin, corticosterone, etc.) and antibiotics (e.g., penicillin, streptomycin, etc.).

In the present invention, as a medium for suspension culture, an S medium may be used. The “S medium” may include DMEM, F12, B27 supplement, bFGF, hEGF, LIF and antibiotics. The S medium may include DMEM and F12 in a ratio of 5 through 1 to 1, though preferably 3 through 1 to 1, B27 supplement at a concentration of 0.1 μl/ml to 1 ml/ml, though preferably 0.1 μl/ml to 100 ul/ml, bFGF at a concentration of 0.1 ng/ml to 1 mg/ml, though preferably 1 ng/ml to 100 μg/ml, hEGF at a concentration of 0.1 ng/ml to 100 μg/ml, though preferably 1 ng/ml to 100 ng/ml, LIF at a concentration of 0.1 ng/ml to 1 mg/ml, though preferably 1 ng/ml to 100 μg/ml, and antibiotics at a concentration of 0.1 μl/ml to 1 ml/ml, though preferably 1 μl/ml to 100 μl/ml.

Subsequently, in step (3), the cells in a suspended state may be recovered from the primary culture. The cells in the suspended sate may be obtained by known methods in the art, for example centrifuging the primary culture, or infiltrating the primary culture using a cell strainer.

In step (4), the cells in a suspended state recovered according to step (3) may be subjected to subculturing a predetermined number of times or more in the medium for suspension culture in a suspended sate, thereby producing a multipotent cancer stem cell line. The medium for suspension culture may be the same as the medium for suspension culture used in step (2). Here, the subculturing may be carried out at least once, preferably at least 5 times, more preferably at least 7 times, and most preferably at least 10 times.

### ADVANTAGEOUS EFFECTS

A multipotent cancer stem cell line according to the present invention can be useful in searching for a novel anticancer drug capable of overcoming resistance to a conventional anticancer drug or developing therapy for maximizing the efficiency of cancer treatment.

### DESCRIPTION OF DRAWINGS

FIG. 1 shows a microscopic image of a multipotent cancer stem cell line according to the present invention, cultured in a suspended state;

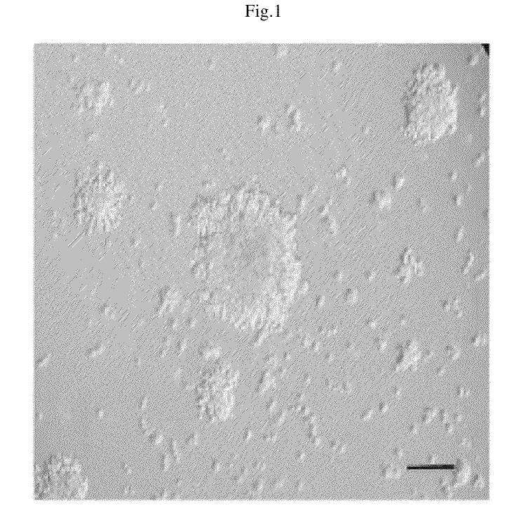

FIG. 2 shows the result of the flow cytometry analysis carried out to examine the expression of a cancer stem cell marker from the multipotent cancer stem cell line according to the present invention;

FIG. 3 shows the results of the immunohistochemistry and confocal microscopy analyses carried out to examine a multipotent cancer stem cell line marker of the multipotent cancer stem cell according to the present invention;

FIG. 4 shows the result of the flow cytometry analysis carried out to analyze a cell cycle of the multipotent cancer stem cell line according to the present invention; and

FIG. 5 shows the tumorigenicity when the multipotent cancer stem cell line according to the present invention is injected into a mouse.

### MODE FOR INVENTION

Hereinafter, the present invention will be described with reference to examples and comparative examples in detail. However, the present invention is not limited to these examples.

### Example 1

### Composition and Preparation of Medium

A medium for suspension culture (S medium) was composed of DMEM:F12=3:1, B27 supplement, 40 ng/ml bFGF, 20 ng/ml EGF, 10 ng/ml LIF, penicillin and streptomycin.

Here, DMEM, F12, B27 supplement, penicillin, streptomycin and trypsin were purchased from Gibco-BRL (Grand Island, N.Y.). EGF, bFGF and LIF were purchased from Invitrogen (Carlsbad, Calif.). A collagenase was purchased from Roche (Indianapolis, Ind.). Fetal bovine serum (FBS) was purchased from HyClone (Cramlington, Northumberland, UK). 40-μm cell strainers, petri dishes, tissue culture dishes and 24-well plates were purchased from Falcon (San Jose, Calif.).

### Example 2

### Production of Multipotent Cancer Stem Cell Line

Breast cancer sarcoma tissue was excised, cut into 1-2 mm pieces, and washed with phosphate-buffered saline (PBS) three times. The resultant tissue was digested in collagenase for 1 hour at 37° C. and treated with FBS to inactivate the collagenase. After enzyme treatment, the resultant tissue was mechanically disrupted by vortexing for several minutes at room temperature. Subsequently, the disrupted tissue was resuspended in S medium by pipetting and then infiltrated using a 40-μm cell strainer. The cells obtained thereby were subjected to centrifugation at 80 G for 5 minutes to collect pellets. PBS was added to the pellets, and centrifugation was carried out twice at 80 G to wash the pellets.

The cells were inoculated into S medium in a petri dish for primary culture in humid air containing 5% CO₂ at 37° C. for 7 days. The primary culture was centrifuged for about 5 minutes to collect suspended cells, after which the cells were inoculated into S medium in a petri dish for subculturing in humid air containing 5% CO₂ at 37° C. The subculturing was carried out every 7 days.

Multipotent cancer stem cell lines obtained through 10× subculturing in a suspended state are referred to as “NDY-1” and were deposited in the Korean Cell Line Research Foundation (KCLRF) on Nov. 17, 2007 (accession No. KCLRF-BP-00174).

### Example 3

### Examination of Expression of Breast Cancer Stem Cell Markers in the Multipotent Cancer Stem Cell Line According to the Present Invention

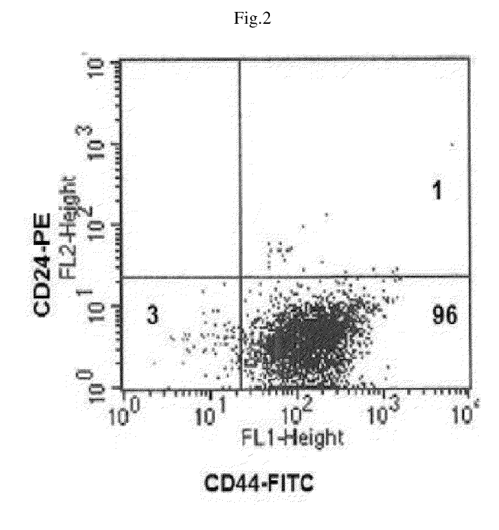

To examine if the multipotent cancer stem cell line obtained by subculturing the cells 10 times in a suspended state according to Example 2 expressed breast cancer stem cell markers, flow cytometry analysis was carried out with an anti-CD24-PE conjugated rat polyclonal IgG antibody (1:10; BD biosciences, NJ, USA) and a CD44-FITC conjugated mouse monoclonal IgG antibody (1:10; BD biosciences, NJ, USA) using FACSCalibur (Becton & Dickinson, San Jose, Calif.) (see FIG. 2).

As shown in FIG. 2, the multipotent cancer stem cell lines according to the present invention include a section expressing only CD24, a section expressing both CD24 and CD44, a section expressing only CD44, and a section expressing neither CD24 nor CD44 in terms of expression of CD24 and CD44, which are breast cancer stem cell markers. On the whole, the multipotent cancer stem cell line shows a CD24^{low/-}CD44^{high} expression pattern, which is characteristic of the breast cancer stem cell marker.

### Example 4

### Analysis of Markers by Immunohistochemical Analysis of the Multipotent Cancer Cell Line According to the Present Invention

The multipotent cancer stem cell line obtained by subculturing the cells at least 10 times in a suspended state according to Example 2 was recovered and centrifuged. After washing with PBS, the multipotent cancer stem cell line was reinoculated on an 18-mm cover slip (Marienfield, Germany) coated with 10 μ/ml of type IV collagen in a 24-well plate containing a medium for attached culture. The cell lines were cultured for 3 days, after which the markers were analyzed.

To examine if the multipotent cancer stem cell line of the present invention expresses an epithelial cell marker, immunohistochemistry and confocal microscopy analyses were carried out. Here, a myoepithelial cell marker was detected using a Vimentin mouse monoclonal IgG3 antibody (1:1000; Chemicon, Temecula, Calif.), and a ductal epithelial cell marker was identified using a Muc1 rabbit polyclonal IgG antibody (1:1000; Calbiochem, Darmstadt, Germany). As secondary antibodies, a rhodamine (TRITC)-conjugated goat anti-rabbit IgG antibody (1:400; ZYMED) and a fluorscein (FITC)-conjugated goat anti-mouse IgG antibody (1:500; Sigma) were used.

Immunohistochemistry analysis was carried out as follows: The multipotent cancer stem cell line cultured on an 18-mm cover slip was washed with PBS and immobilized with 4% formaldehyde solution for 15 minutes at room temperature. Subsequently, the immobilized cell line was washed with PBS three times and subjected to penetration with 0.5% Triton X-100 for 15 minutes at room temperature, followed by washing with PBS three times. The cell lines were blocked using 5% FBS-supplemented PBS for 1 hour at room temperature and then washed with PBS several times. The cells were maintained with primary antibody for 1 hour. The cells were then washed with PBS several times, maintained with secondary antibody for 1 hour, and then washed with PBS three times. The immunohistochemically stained cells were stored in Vectashield medium (Vector Laboratories, Burlingame, Calif.) on which 4′6′-diamidino-2-phenylindole hydrochloride (DAPI) was stacked.

Subsequently, confocal microscopy was carried out using an Axiovert LSM 510 microscope (Zeiss, Jena, Germany). The obtained images were processed using LSM Pascal ver. 3.1 and Photoshop 7.0 (Adobe Systems, San Jose, Calif.). Double-labeled samples having FITC- and/or TRITC-conjugated secondary antibodies were simultaneously or sequentially analyzed. In each case, FITC was excited with a blue beam and detected using an interferential narrow band filter (BP 505-550 nm), whereas TRITC was excited with a red beam and detected using a long pass filter (LP 650 nm).

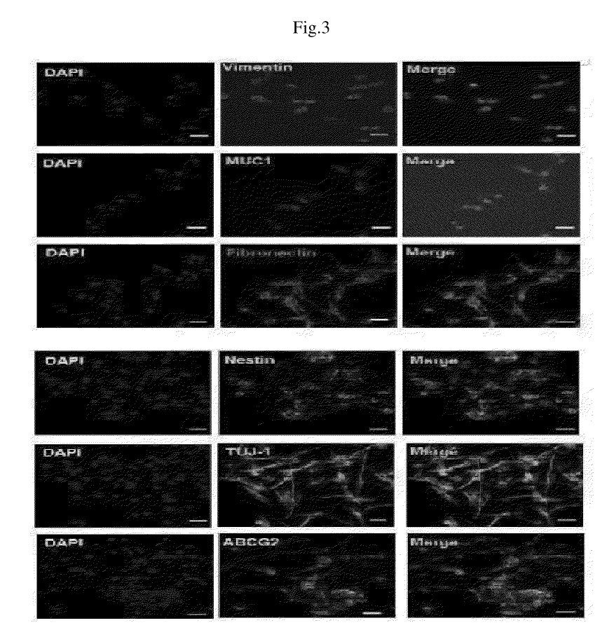

As shown in FIG. 3, the multipotent cancer stem cell line was labeled with anti-Vimentin (green) and anti-Muc1 (red). The nuclei of the cells were stained with DAPI (blue). From these results, it can be noted that the multipotent cancer stem cell line according to the present invention is capable of being differentiated into myoepithelial cells and ductal epithelial cells.

To examine if the multipotent cancer stem cell line according to the present invention expresses an anticancer drug resistance protein and a neural cell marker, immunohistochemistry and the confocal microscopy were performed. The anticancer drug resistance protein was detected using a mouse monoclonal IgG1 anti-ABCG2 antibody (1:100; BD Pharmingen), and a neural stem cell/progenitor cell marker was identified with a mouse monoclonal IgG1 anti-nestin antibody (1:100; BD Pharmingen) and mouse monoclonal IgG1 anti-Tuj-1 antibody (1:500; Sigma).

Immunohistochemistry and confocal microscopy were carried out according to the same procedures as described above.

As shown in FIG. 3, the multipotent cancer stem cell line was labeled with DAPI for staining of nuclei (blue), anti-ABCG2 (green), anti-nestin (green) and anti-Tuj-1 (green). It can be seen that the multipotent cancer stem cell line according to the present invention can express the anticancer drug resistance protein and be differentiated into a neural cell.

Moreover, immunohistochemistry and confocal microscopy were used to examine if the multipotent cancer stem cell line of the present invention expresses a mesenchymal cell marker. The mesenchymal cell marker was identified with a fibronectin rabbit polyclonal IgG antibody (1:1000; Sigma).

Here, immunohistochemistry and confocal microscopy were carried out according to the same procedures as described above.

As shown in FIG. 3, the multipotent cancer stem cell line was labeled with DAPI for staining of nuclei (blue) and anti-fibronectin (red). It can be seen that the multipotent cancer stem cell line according to the present invention can express a mesenchymal cell marker and be differentiated into a mesenchymal cell.

### Example 5

### Analysis of Cell Cycle of the Multipotent Cancer Stem Cell Line According to the Present Invention

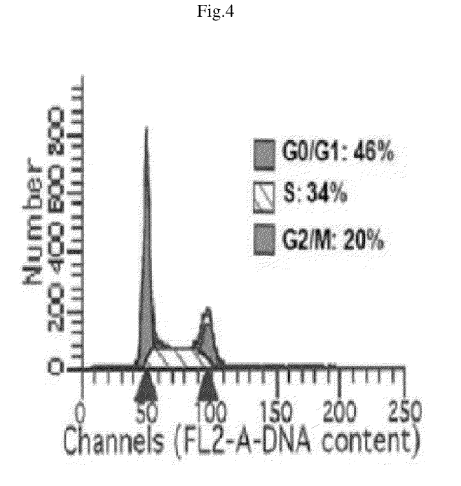

To analyze a the cycle of the multipotent cancer stem cell line according to the present invention, the multipotent cancer stem cell line obtained by subculturing the cells at least 10 times in a suspended state according to Example 2 was treated with trypsin-EDTA to isolate single cells, after which the cells were immobilized with 70% ethanol for 1 hour. Subsequently, the cells were treated with 100 μg/ml of RNase A and activated in a 37° C. incubator for 1 hour. After being treated with 25 μg/ml of a propidium iodide solution, flow cytometry analysis was carried out using FACSCalibur (Becton & Dickinson, San Jose, Calif.) (See FIG. 4).

As shown in FIG. 4, in the suspension culture of the multipotent cancer stem cell according to the present invention, the cells in G1/G0, S and G2/M phases of the cell cycle constituted 46%, 34% and 20% of the total, respectively. Considering the cell cycle of hematopoietic cells in which cells in G1/G0 phases constitute 99%, it can be seen that the multipotent cancer stem cell line according to the present invention has a different cell cycle from those of common stem cells.

### Example 6

### Analysis of Tumor Formation from the Multipotent Cancer Stem Cells According to the Present Invention

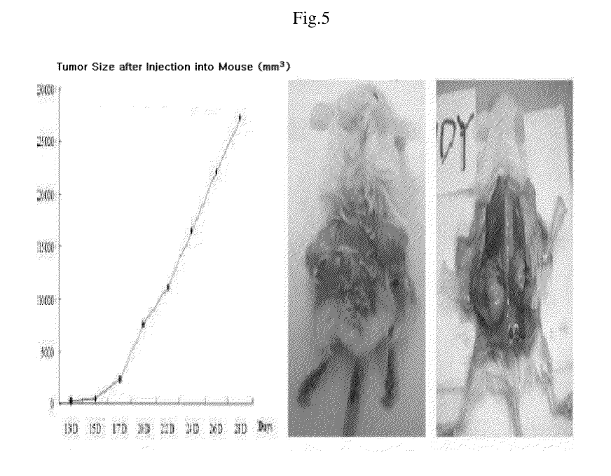

The multipotent cancer stem cell line obtained by subculturing the cells at least 10 times in a suspended state according to Example 2 was washed with PBS twice. Subsequently, after the cells were treated with trypsin-EDTA for about 5 minutes and examined for detachment from a tissue culture dish using a microscope, a medium for attached culture was inoculated in order to inactivate trypsin. The cells were centrifuged for 5 minutes, and pipetting was carried out so that the collected cells became single cells, which were then stained with a 0.4% trypan blue stain solution (Gibco-BRL, Grand Island, N.Y.) to count the number of living cells. The cells were grown until the number of cells in 2 mg/ml of mitrigel (BD Pharmingen, NJ, USA) reached 500,000 and then injected into a mouse. After injection into the mouse, tumor formation was observed every 3 days. Two weeks after the injection, tumor formation could be observed (see FIG. 5). It was concluded that the multipotent cancer stem cell line of the present invention has tumorigenicity.

1. A multipotent cancer stem cell line derived from breast cancer tissue and expressing a breast cancer stem cell marker.

2. The cell line according to claim 1, wherein the breast cancer tissue is a sarcoma.

3. The cell line according to claim 1, wherein the breast cancer stem cell marker is CD24^{low/-}CD44^{high}.

4. The cell line according to claim 1, which expresses at least one selected from the group consisting of an epithelial cell marker, a neural cell marker and a mesenchymal cell marker.

5. The cell line according to claim 4, wherein the epithelial cell markers are Vimentin and Muc1.

6. The cell line according to claim 4, wherein the neural cell markers are nestin and Tuj-1.

7. The cell line according to claim 4, wherein the mesenchymal cell marker is fibronectin.

8. The cell line according to claim 1, which expresses an anticancer drug resistance protein.

9. The cell line according to claim 8, wherein the anticancer drug resistance protein is ABCG2.

10. The cell line according to claim 1, which is cultured in a suspended state.

11. The cell line according to claim 1, which is deposited under Accession No. KCLRF-BP-00174.

12. A method for producing a multipotent cancer cell line, comprising:
(1) isolation of a breast cancer cell from previously extracted breast cancer tissue;
(2) primary culture of the isolated breast cancer cell in a suspended state in a medium for suspension culture;
(3) recovery of cells in a suspended state from the primary culture; and
(4) production of a multipotent cancer stem cell line by subculturing the recovered cells a predetermined number of times or more in a suspended state in the medium for suspension culture.

13. The method according to claim 12, wherein the medium for suspension culture includes DMEM and F12 in a ratio of 1 through 3 to 1, B27 supplement at a concentration of 0.1 μl/ml to 1 ml/ml, bFGF at a concentration of 0.1 ng/ml to 1 mg/ml, hEGF at a concentration of 0.1 ng/ml to 100 ng/ml, LiF at a concentration of 0.1 ng/ml to 1 mg/ml, and an antibiotic at a concentration of 0.1 μl/ml to 1 ml/ml.
